(19) United States
(12) Patent Application Publication
(10) Pub. No.: US 2025/0312558 A1
(43) Pub. Date: Oct. 9, 2025

(54) TREATMENT OF INSOMNIA USING AUDITORY FEEDBACK

(71) Applicant: BELL THERAPEUTICS INC., Seoul (KR)

(72) Inventors: Jonghwa PARK, Seoul (KR); Wonwoo LEE, Goyang-si (KR); Jaeeun LEE, Seoul (KR); Jiyeon HA, Yangju-si (KR); Joongbae YUM, Seoul (KR); Bumjoon KIM, Seoul (KR)

(21) Appl. No.: 19/009,414

(22) Filed: Jan. 3, 2025

Related U.S. Application Data
(63) Continuation-in-part of application No. PCT/KR2024/011122, filed on Jul. 30, 2024.

(30) Foreign Application Priority Data
Jul. 31, 2023 (KR) 10-2023-0099466
Jan. 5, 2024 (KR) 10-2024-0002224
May 10, 2024 (KR) 10-2024-0061711

(51) Int. Cl. A61M 21/02; A61M 21/00

(52) U.S. Cl. CPC A61M 21/02; A61M 2021/0027; A61M 2021/005; A61M 2021/0077; A61M 2021/0088; A61M 2205/505; A61M 2230/63

(57) ABSTRACT

The present invention relates to a method for treatment insomnia using auditory feedback. The treatment method according to the present invention involves identifying the periodicity of the patient's biological signs (e.g., respiration) based on the patient's intentional activity and providing a sound corresponding to the periodicity of the biological signs. This enables accurate real-time synchronization of feedback stimuli such as the cycle of biological signs and the sound, allowing for the induction of slower breathing and/or synchronization with other biological signs (e.g., brainwaves). It shows excellent effects in shortening the patient's sleep onset time and improving the quality of sleep, making it a useful insomnia treatment method. Moreover, the treatment method of the present invention minimizes side effects, enables natural sleep induction, and offers high accessibility without incurring significant costs, making it widely applicable to various users.

### CROSS-REFERENCE TO RELATED APPLICATION(S)

This application is a Continuation-In-Part (CIP) application, claiming priority under 35 U.S.C. § 365(c), of an International application No. PCT/KR2024/011122, filed on Jul. 30, 2024, which is based on and claims the benefit of a Korean patent application number 10-2023-0099466, filed on Jul. 31, 2023, in the Korean Intellectual Property Office, of a Korean patent application number 10-2024-0002224, filed on Jan. 5, 2024, in the Korean Intellectual Property Office, and of a Korean patent application number 10-2024-0061711, filed on May 10, 2024, in the Korean Intellectual Property Office, the disclosure of each of which is incorporated by reference herein in its entirety.

### TECHNICAL FIELD

The present disclosure relates to a method for treatment insomnia using auditory feedback.

### DESCRIPTION OF RELATED ART

Insomnia refers to a condition in which a person cannot achieve normal sleep, spending most of the day in a state of wakefulness (sleeplessness), or experiencing significantly insufficient sleep even when they do sleep. Symptoms include being unable to fall asleep despite prolonged wakefulness and fatigue, or waking up soon after falling asleep. Conventional sleeping pills have a central depressant effect similar to anesthetics. In small doses, they have a calming effect, in moderate doses, they induce sleep, but in large doses, they can cause coma, paralysis, and respiratory depression. Barbiturate drugs have low safety, and tolerance and dependence can easily develop. After long-term use, discontinuation can lead to insomnia due to nightmares and other issues.

Meanwhile, content for sleep induction has recently been actively developed. For example, such content may include content for sleep induction or content effective for sleep induction (e.g., audio and/or graphics). For instance, U.S. Pat. No. 10,576,355 B2 discloses a configuration that induces the durations of the user's respiratory phases (e.g., inhale/exhale, etc.) to achieve a desired duration ratio by providing stimuli that change over time. In other words, conventional methods for sleep induction provide stimuli (e.g., sound) that are set to reach specific goals (e.g., an ideal inhale/exhale duration ratio) determined by a system (or algorithm).

The applicants, while researching methods to treat insomnia, discovered that a method involving the steps of instructing the patient to perform a first activity and inhale, detecting the patient's first activity, and providing a first sound; and instructing the patient to perform a second activity and exhale, detecting the patient's second activity, and providing a second sound, was highly effective in treating insomnia. This led to the completion of the present invention.

The present invention is conceived to solve the aforementioned problems, and its objective is to provide a method for treatment insomnia using auditory feedback. The problems addressed by the present disclosure are not limited to those mentioned above, and other problems not explicitly mentioned will be clearly understood by those skilled in the art from the descriptions below.

### SUMMARY

To solve the technical problem, the present invention provides a method for treatment insomnia comprising:
(i) instructing the patient to perform a first activity and inhale, detecting the patient's first activity, and providing a first sound;
(ii) instructing the patient to perform a second activity and exhale, detecting the patient's second activity, and providing a second sound;
(iii) alternately repeating steps (i) and (ii) until at least one condition is satisfied;
(iv) providing the first sound independently of detecting the patient's activity;
(v) providing the second sound independently of detecting the patient's activity; and
(vi) alternately repeating steps (iv) and (v).

The method for treatment according to the present invention involves confirming the periodicity of the patient's biological signs (e.g., breathing) based on the patient's intentional activity and providing a sound corresponding to the periodicity of the biological signs. This allows for the precise real-time synchronization of feedback stimuli such as the periodicity of biological signs and sound. It can induce slower breathing and/or synchronize biological signs with other biological signs (e.g., brain waves), leading to a reduction in the patient's sleep onset time and improvement in sleep quality. Thus, it can be effectively used as a method for treatment insomnia. Furthermore, the method for treatment according to the present invention minimizes side effects, enables natural sleep induction, and offers high accessibility without incurring additional costs, making it widely applicable to various users.

The advantages of the present invention are not limited to those mentioned above, and other advantages not explicitly described will be clearly understood by those skilled in the art from the descriptions below.

### BRIEF DESCRIPTION OF THE DRAWINGS

A more complete appreciation of the disclosure and many of the attendant aspects thereof will be readily obtained as the same becomes better understood by reference to the following detailed description when considered in connection with the accompanying drawings, wherein:

FIG. 1 is a schematic representation of the method for treatment insomnia according to the present invention.

FIG. 2 shows a screen provided during insomnia treatment according to one embodiment.

FIGS. 3A to 3E illustrate the relationship between the patient's activity and the provided sound in the method for treatment according to the present invention.

FIG. 4 is a schematic representation of the sleep induction experiment process for insomnia patients.

FIGS. 5A and 5B show the screens provided to the subjects during the sleep induction experiment.

FIGS. 6A to 6E visualize the polysomnography results according to the use of the method for treatment of the present invention.

FIGS. 7A to 7D visualize the results of the K-RCSQ test (depth of sleep, time to fall asleep, awakenings during sleep, waking up during sleep, overall sleep quality) depending on the use of the method for treatment of the present invention.

FIG. 8 shows the sensor attachment locations for EEG testing during insomnia treatment according to the present invention.

FIG. 9 shows a photograph of the EEG testing experiment conducted during insomnia treatment according to the present invention.

FIG. 10 shows the EEG measurement results depending on the use of the method for treatment of the present invention.

FIG. 11 shows the average change in EEG when using the method for treatment of the present invention compared to EEG when using sleeping pills.

FIG. 12 is a schematic representation of the sleep induction experiment process for breast cancer patients.

FIG. 13 visualizes the average K-RCSQ scores evaluated on the morning of the surgery day and the morning after the surgery.

### DETAILED DESCRIPTION

Hereinafter, the preferred embodiments of the present invention will be described in detail with reference to the accompanying drawings. The advantages and features of the present invention, as well as the methods for achieving them, will become apparent by referring to the embodiments described below along with the attached drawings. However, the present invention is not limited to the embodiments presented hereinafter and can be implemented in various forms. These embodiments are provided merely to ensure the completeness of the description of the invention and to fully inform those skilled in the art of the scope of the invention, and the present invention is defined only by the scope of the claims. Throughout the specification, the same reference numerals refer to the same components.

Unless otherwise defined, all terms used in this specification (including technical and scientific terms) are used in a manner that would be commonly understood by those skilled in the art to which the invention pertains. Terms that are generally defined in dictionaries will not be interpreted in an excessively broad or narrow sense, unless explicitly defined otherwise. The terms used in this specification are intended to describe the embodiments and are not meant to limit the invention. In this specification, singular forms include plural forms unless specifically stated otherwise.

The terms “comprises” and/or “comprising” used in this specification do not exclude the presence or addition of one or more other components, steps, operations, and/or elements beyond those mentioned.

First, the present invention provides a method for treatment insomnia comprising:
(i) instructing the patient to perform a first activity and inhale, detecting the patient's first activity, and providing a first sound;
(ii) instructing the patient to perform a second activity and exhale, detecting the patient's second activity, and providing a second sound;
(iii) alternately repeating steps (i) and (ii) until at least one condition is satisfied;
(iv) providing the first sound independently of detecting the patient's activity;
(v) providing the second sound independently of detecting the patient's activity; and
(vi) alternately repeating steps (iv) and (v).

The at least one condition is satisfied when at least one of the following conditions (a) to (f) is met.
(a) failing to detect at least one of the patient's first or second activities;
(b) detecting a third activity designated for entry into step (iv);
(c) detecting that the duration of at least a portion of the patient's first or second activity exceeds a specified threshold length;
(d) confirming that the difference between the patient's first and second activity intervals is less than a critical difference;
(e) confirming that at least one measured biological sensing data from the patient satisfies a specified rule; and
(f) elapsed time exceeding a specified period since the initiation of step (i).

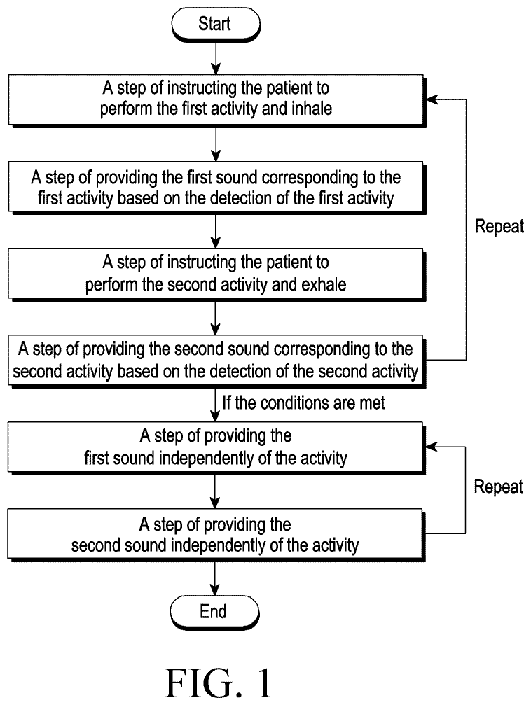

The method for treatment insomnia according to the present invention is schematically shown in FIG. 1, and will be described in detail below.

In step (i), the patient is instructed to perform a first activity while inhaling, the patient's first activity is detected, and a first sound is provided. In step (ii), the patient is instructed to perform a second activity while exhaling, the patient's second activity is detected, and a second sound is provided. The first activity and second activity are activities that are independent of the inhalation or exhalation, not activities caused by the breath (inhalation or exhalation).

Preferably, the first activity may be an activity that causes tension in at least some of the patient's muscles, and the second activity may be an activity that causes relaxation of at least some of the patient's muscles.

As an example embodiment, the first activity may be set as a “tap-on event” (for instance, an event where the patient touches a touchscreen), and the second activity may be set as a “tap-off event” (for instance, an event where the patient releases their touch from the touchscreen). For example, when a tap-on event occurs, pressure may be applied between the patient's finger and the surface of the touchscreen, which could cause tension in the patient's finger (or muscles near the finger). On the other hand, when a tap-off event occurs, the tension previously caused in the patient's finger (or the surrounding muscles) may be relieved (i.e., muscle relaxation). Muscle relaxation is helpful for sleep induction; however, requiring the patient to relax their muscles unilaterally may be less effective than temporarily tensing the muscles and then requesting relaxation, as this method may more effectively induce sleep. Muscle relaxation may also reduce the activation level of the arousal system originating from the amygdala, thereby enhancing the sleep-inducing effect.

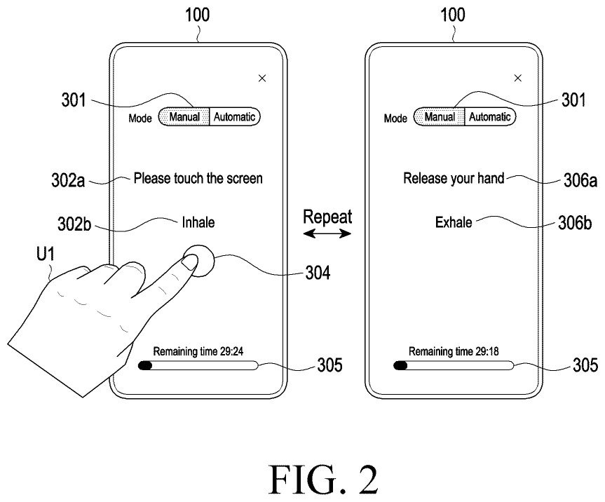

An exemplary screen provided during insomnia treatment in the present invention is shown in FIG. 2. As shown in FIG. 2, the first activity may be set to occur based on a touch (304) by the subject, and the second activity may be set to occur based on the release of the touch (304).

In this context, inhalation refers to the process of air entering the lungs from the external environment, while exhalation refers to the process of air leaving the lungs to the external environment. Inhalation and exhalation occur cyclically and repeat. The steps (i) and (ii) provide sound during at least some portion of the time during inhalation or exhalation.

Since the sound is provided based on the activity detection of the sensor unit, the start and end points of the provision of the first sound are substantially synchronized with the start and end points of the detection of the first activity, and the start and end points of the provision of the second sound are substantially synchronized with the start and end points of the detection of the second activity.

The end point of the first sound is set after the end point of the first activity, and by providing the first sound for a period longer than the detection period of the first activity, the patient can be induced to perform a longer inhalation. Similarly, the end point of the second sound is set after the end point of the second activity, and by providing the second sound for a period longer than the detection period of the second activity, the patient can be induced to perform a longer exhalation. Based on this asymmetric synchronization of the start and end points, slow breathing can be induced. Depending on the synchronization of the start point, the patient's neurons, brainwaves, and/or attention may become synchronized with the sound. As a result, the patient may also synchronize with the end effect, which can further slow down the breathing. Slow breathing activates the parasympathetic nervous system, and activation of the parasympathetic nervous system can enhance the sleep-inducing effect. Additionally, slow breathing can induce hyperpolarization, which can further enhance the sleep-inducing effect. Moreover, slow breathing may reduce the impact on the amygdala, thereby decreasing the activation potential of the arousal system originating from the amygdala and increasing the sleep-inducing effect.

There are no limitations on the method of activity detection. As an example embodiment, the first activity can be detected by confirming the occurrence of the first activity based on at least one sensing data, and the second activity can be detected by confirming the occurrence of the second activity based on at least one sensing data. For example, based on sensing data (or processing results) from a touchscreen, both the tap-on event and the tap-off event can be detected. Specifically, the occurrence of the first activity can be confirmed based on the occurrence of a tap-on event once or a predefined number of consecutive occurrences, and the occurrence of the second activity can be confirmed based on the detection of a tap-off event. Alternatively, the second activity can be confirmed to have occurred based on the detection interruption of the event corresponding to the first activity (e.g., the tap-on event), or by detecting a failure to detect, a failure to meet detection conditions, or an undetected occurrence of the first activity.

In the present invention, the sound may also be referred to as an auditory content, auditory stimulus, or auditory feedback, and due to its ability to vibrate the eardrum of the patient and/or cause entrainment in at least a portion of the body, it may also be referred to as a physical feedback.

The first sound and the second sound are different sounds, and preferably, they may have at least a half-tone difference, and more preferably, at least a full-tone difference. Specifically, the frequency (f1) of the first sound and the frequency (f2) of the second sound may satisfy the following Equation 1. Here, f1 and f2 refer to the number of vibrations per unit time, with the unit being Hertz (Hz).

$$f 1 : f 2 = m 2 a : n 2 b \qquad Equation 1$$

Here, m and n are independently 1, 3, or 5, and a and b are independently integers between 0 and 8, with the condition that when m=n, a≠b.

Additionally, the first sound and the second sound are preferably related by a perfect octave, perfect fifth, perfect fourth, major third, major sixth, minor third, or minor sixth.

The first sound and the second sound may each be provided in such a way that the volume gradually increases from the start point of the sound to the point of maximum volume, and then gradually decreases from the maximum volume point to the endpoint of the sound.

The time from the start point of the first sound and the second sound to their respective maximum volume point may be shorter than the time from the maximum volume point to the endpoint of the sound. Preferably, the time from the start point to the maximum volume point for each of the first sound and the second sound may be no more than 75% of the time from the maximum volume point to the endpoint of the sound. More preferably, the time from the start point to the maximum volume point for each of the first sound and the second sound may be no more than 50% of the time from the maximum volume point to the endpoint of the sound.

The first sound and the second sound may fade out at the endpoint of their provision. Fade-out is a technique used in music where the sound gradually diminishes and eventually disappears, with the volume slowly decreasing until it is completely gone.

The fade-out of the first sound may be performed such that the first sound ends after the start of the second sound, which is provided immediately after the first sound. Similarly, the fade-out of the second sound may be performed such that the second sound ends after the start of the first sound, which is provided immediately after the second sound.

The first sound may include a plurality of first sub-sounds, and the second sound may include a plurality of second sub-sounds. Specifically, the first sound may be a composite sound formed by the sum of sub-sounds at various frequencies.

The first sound and the second sound may be expressed as sounds in musical form, such as instrument sounds, electronic sounds, MIDI sounds, or special effect sounds, but are not limited to these. For example, the sounds may also imitate natural sounds or human breathing sounds (for instance, sounds imitating the breathing of the patient or someone else, such as the voice of the patient or another person). The “human” may refer to the patient, family, acquaintances, or even a public figure, and is not limited to these.

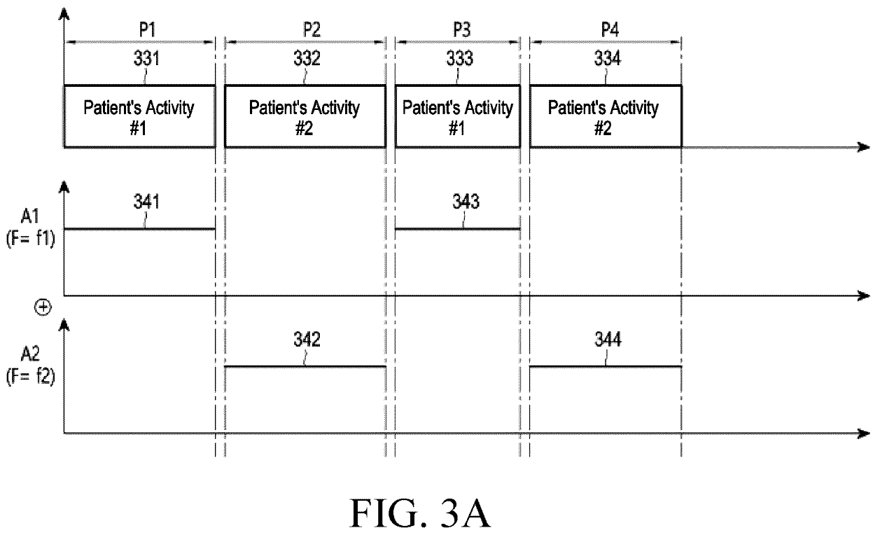

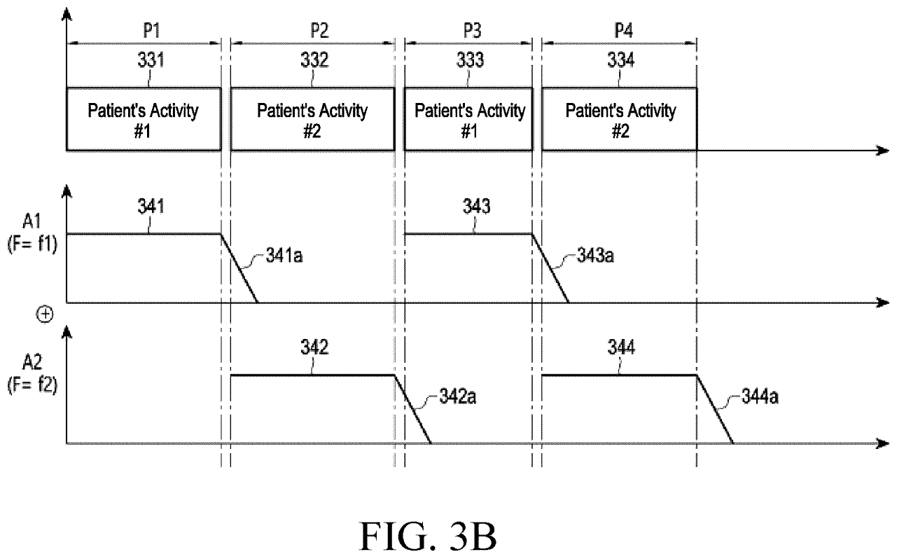

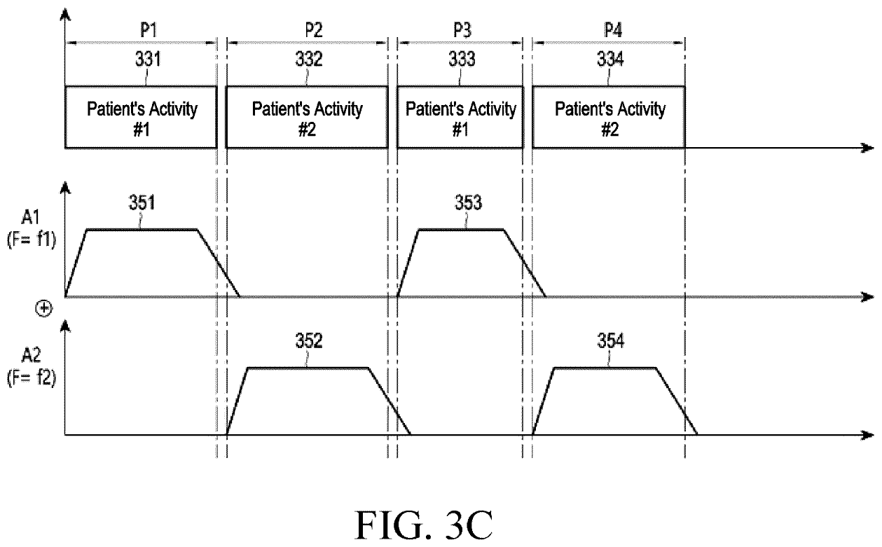

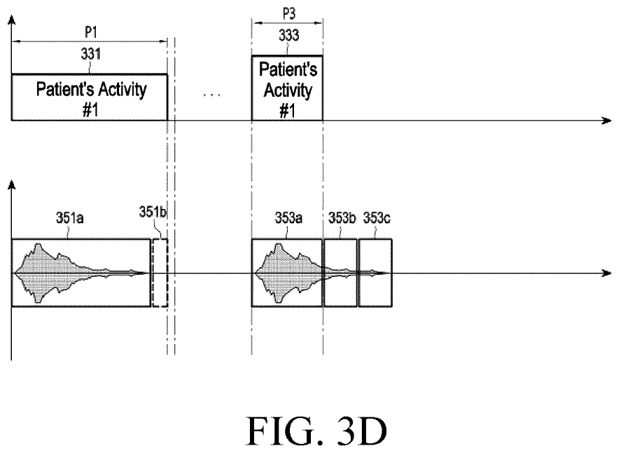

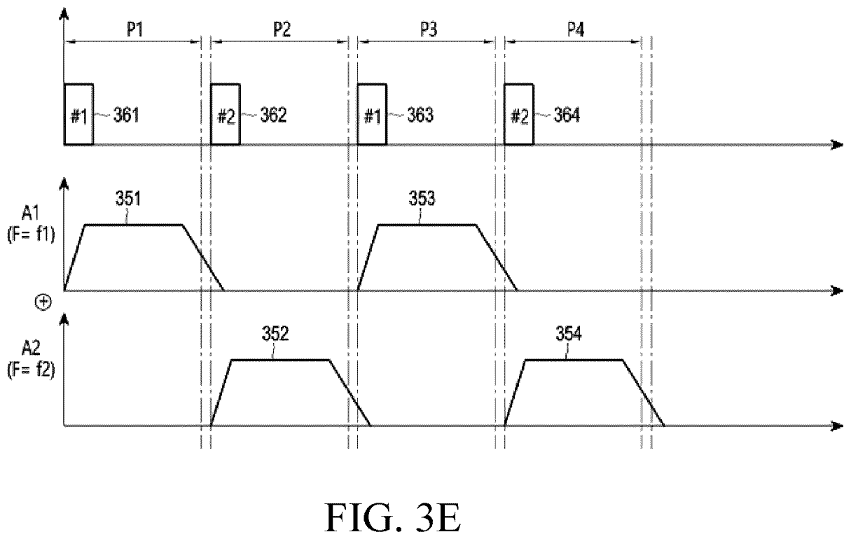

In the present invention, the relationship between the patient's activity and the provided sounds is exemplified in FIGS. 3A to 3E. As shown in FIG. 3A, the first sound (341, 343) and the second sound (342, 344) may be alternately provided. For example, during the first period (P1), the occurrence (or maintenance) of the patient's first activity (331) is confirmed, and based on the occurrence (or maintenance) of the first activity (331), the first sound (341) corresponding to the first activity (331) may be provided. In FIG. 3A, for example, the first sound (341) may have a constant amplitude (e.g., A1) and a single frequency (e.g., f1), but is not limited to this.

In the example of FIG. 3A, for instance, the provision of the first sound (341) may be stopped based on the cessation of the first activity (331), but there is no limitation on the trigger for stopping the provision of the first sound (341). For example, the detection of the second user activity 332 could serve as a trigger to stop providing the first sound 341. Meanwhile, the cessation of the first sound 341 may, for example, refer to the immediate termination of the output of the first sound 341 or the application of an ending effect (e.g., a fade-out effect), as would be understood by a person skilled in the art. For instance, the provision timing of the first sound 341 may be substantially synchronized with the detection timing of the first user activity 331. Similarly, the cessation timing of the first sound 341 may be substantially synchronized with the timing of the cessation trigger (e.g., the detection failure of the first user activity 331 or the detection of the second user activity 332), or it may not be substantially synchronized.

Meanwhile, the occurrence (or maintenance) of the patient's second activity (332) during the second period (P2) can be confirmed. Based on the occurrence (or maintenance) of the second activity (332), a second sound (342) corresponding to the second activity (332) can be provided. In FIG. 3A, by way of example, the second sound (342) may have a constant amplitude (e.g., A2) and a single frequency (e.g., f2), but it is not limited to this.

In FIG. 3A, by way of example, the amplitude of the second sound (342) may change and/or its frequency may change, and its waveform is not limited. The characteristics of the second sound (342) (which may include amplitude and/or frequency, but are not limited to these) may be the same as or at least partially different from the characteristics of the first sound (341).

In FIG. 3A, by way of example, the provision of the second sound (342) may be stopped based on the cessation of the second activity (332), but there is no limitation on the trigger for stopping the provision of the second sound (342).

As shown in FIG. 3B, in contrast to FIG. 3A, during the provision of the first sound (341), based on the confirmation of the end (or failure) of the detection of the first activity (331), the first sound (341) with an applied end effect (341a) may be provided.

As one example, the first sound (341a) with an applied end effect may be a sound whose amplitude decreases over time (for instance, this could be referred to as a fade-out), but there is no limitation on the type and/or number of the end effects. On the other hand, the end (or failure) of the detection of the first activity (331) is merely one example of a trigger for applying the end effect.

As another example, the end of the detection of the first activity (331) may not be the trigger, and instead, the detection of the second activity (332) may serve as the trigger for providing the first sound (341a) with an applied end effect. Alternatively, the passage of a specified period of time may serve as the trigger for applying the end effect.

As shown in FIG. 3B, the start point of the detection of the first activity (331) may be substantially synchronized with the start point of the provision of the first sound (341). Additionally, the end point of the detection of the first activity (331) (or the start point of the detection of the second activity (332)) may be substantially synchronized with the start point of the end effect of the first sound (341).

As shown in FIG. 3C, the first sound (351) corresponding to the first activity (331) and the second sound (352) corresponding to the second activity (332) may be provided. In contrast to FIG. 3A, where the first sound (341) has a substantially constant amplitude, the first sound (351) in FIG. 3C may be composed of three parts: the first part, where the amplitude increases over time, the second part, where the amplitude is maintained over time, and the third part, where the amplitude decreases over time.

Meanwhile, the provision of the sounds (351, 352, 353, 354) may continue even after the activities (331, 332, 333, 334) have ended. For example, the detection timing (or the confirmation timing) of the patient's activities (331, 332, 333, 334) may be substantially synchronized with the timing of the provision of the sounds (351, 352, 353, 354) (i.e., their start points are substantially synchronized), while the provision end time of the sounds (351, 352, 353, 354) may differ from the end point of the detection (or detection termination) of the activities (331, 332, 333, 334) (i.e., their end points are set differently). As a result, in some sections, the sounds (351) and (352) may overlap in their provision (or, the combined result of sounds (351) and (352) may be provided).

As shown in FIG. 3D, a sound (351a) with a waveform corresponding to the first activity (331) may be provided. The waveform of the sound (351a) can, for example, be a waveform where the amplitude increases and then decreases, but it is not limited to this. At least one characteristic of the waveform (or envelope), such as attack, decay, sustain, and release (though not limited to these), may be implemented to correspond to the breathing characteristics, but this is not restrictive.

The attack refers to the time it takes for the sound to initially form and reach its maximum level. During the attack phase, the sound increases sharply, determining how the sound begins. The decay is the time it takes for the sound to drop from the maximum level to the sustain level after the attack phase. During decay, the sound decreases and reaches the sustain level. The sustain represents the duration of the sound, during which the sound's level is maintained at a steady state. The release is the time it takes for the sound to gradually fade away when the key is released, with the sound decreasing slowly until it disappears naturally.

Breathing characteristics may include movements of the chest during breathing, airflow through the respiratory tract (e.g., through the nose), or derivatives such as the differential or integral of airflow (though not limited to these). The types and number of characteristics are not restricted. The sound (351a) can consist of a first part (or beginning) where the amplitude increases and a second part (or ending) where the amplitude decreases, but the waveform of the sound (351a) is not limited to this form. The sound (351a) may have a specified playback duration (which could be a fixed value or changeable based on cumulative instances, for example).

Therefore, if the first activity (331) is detected beyond the playback duration of the sound (351a), the sound provision may be stopped during the remaining part (351b). However, this is just an example, and a sound corresponding to the reverb of sound (351a) may be provided during the remaining part (351b), and the sound that can be played in the remaining section (351b) is not restricted.

Additionally, the duration (P3) of the first activity (333) may be shorter than the specified playback duration. If the detection of the first activity (333) is interrupted during the provision of the first part (353a) of the designated waveform, the interruption can trigger the application of an end effect (e.g., a fade-out effect, though not limited to this) to the second part (353b). The duration of the application of the end effect may be pre-set, and after the elapsed period of the end effect, the third part (353c) may not be provided.

As shown in FIG. 3E, based on the detection of the first activity (361), which is associated with the patient's awareness of the first part of the first biometric signal (e.g., the inhalation phase of breathing), the first sound (351) can be provided. Similarly, based on the detection of the second activity (362), which is associated with the patient's awareness of the second part of the first biometric signal (e.g., the exhalation phase of breathing), the second sound (352) can be provided.

For example, even if the first activity (361) is not maintained, the first sound (351) can still be provided based on the detection of the first activity (361). Likewise, based on the detection of activities (362, 363, 364), the corresponding sounds (352, 353, 354) can be provided. While sounds (351, 352, 353, 354) are shown with decreasing amplitude over a specified time period, this is just an example. For instance, the amplitude of the first sound (351) corresponding to the first activity (361) may decrease based on the detection of the second activity (362) as a trigger.

In another example, each sound could be implemented with a specified waveform. For example, based on the detection of activities (361, 362, 363, 364), each sound (351, 352, 353, 354) with a fixed playback duration could be provided, and this could be applied to other embodiments as well.

The steps (i) and (ii) may involve providing content related to bio-information along with the sound. The content associated with the bio-information can be based on biofeedback or bio-sonification, which are related to the physiological data. Specifically, based on the patient's activity, the periodicity of a specific biometric signal can be identified, and content that is substantially synchronized with the identified periodicity can be provided.

From the patient's perspective, based on their activity in relation to the periodicity of their physiological signs, they can experience content that is practically synchronized with their periodicity. This process can be referred to as biofeedback. On the other hand, content can also be implemented through sound, and the sound that is practically synchronized with the physiological signs can be referred to as bio-sonification.

For example, in terms of biofeedback, the physiological processes of the body can be monitored in real-time, and content related to this information can be provided. Here, the physiological processes can include not only periodic physiological signs such as breathing (inhalation and exhalation), heart rate, and blood pressure, but also non-periodic physiological signs like muscle tension and skin temperature, though this is not limited to them.

For example, a UI (user interface) can be provided to guide the patient's perception of vital signs with periodicity. At least one activity can be acquired to represent the patient's perception of the vital signs, and at least one content corresponding to each activity can be provided. The patient can experience at least one of these contents.

Based on the detection of activities related to the patient's perception of vital signs, the periodicity of the vital signs can be confirmed, allowing for the provision of content that is practically synchronized with the periodicity without performing detailed physiological signal analysis. Furthermore, since the patient must perceive the periodicity of the vital signs to perform the activity, their focus on the recognition of the periodicity can be enhanced, potentially leading to slower breathing and/or early sleep onset.

Additionally, based on the biofeedback-driven content experience, the patient's focus on recognizing the periodicity of vital signs can be further enhanced, and other physiological signs of the patient (e.g., brainwaves, though not limited to them) can synchronize with the periodic vital signs. If the periodic vital sign is the breath cycle (inhalation and exhalation), other physiological signs, such as brainwaves (or specific brain areas), can be synchronized with the content. As a result, the breathing cycle and brainwaves (or specific brain areas) can synchronize, potentially leading to relatively faster sleep onset.

In the above (iii), the steps (i) and (ii) can alternately repeat until at least one condition is met, and the first sound and the second sound are repeatedly provided in response to the patient's first and second activities.

In (iii), during the repetition of steps (i) and (ii), at least one property of the first sound and the second sound may change or remain the same. That is, the first sound corresponding to the first activity may be the same as or at least partially different from the first sound of the previous cycle, and the second sound corresponding to the second activity may be the same as or at least partially different from the second sound of the previous cycle.

The attributes of the sound to be changed can be determined based on the biological sensing data acquired during the previous cycle. The determination (or modification) of the sound attributes based on the biological sensing data can be performed using at least one rule-based system, and/or may be based on the inference results of at least one artificial intelligence model.

The biological sensing data may include, but is not limited to, RR (respiratory rate), HR (heart rate), HRV, brainwaves, stress level, sounds corresponding to breathing sounds, and/or muscle tension.

The attributes may include, but are not limited to, volume, waveform, frequency, and timbre.

The at least one condition may be any of the following (a) to (f), and when at least one of these conditions is satisfied, the (iv) step is performed.
(a) The condition is when the detection of at least one of the patient's first or second activities fails, with the failure detection time being practically synchronized with the start time of the first sound in step (iv). Since the patient's activity detection has failed, sound is provided independently of the activity detection.
(b) The condition is when a third activity, designated to enter the (iv) step, is detected. This third activity is predefined to provide sound independently of the patient's activity detection and is not limited to a specific activity.
(c) The condition is when it is detected that at least some portion of the duration of the patient's first or second activity is longer than a specified threshold length. This threshold length may be set, for example, to a length that is effective for sleep onset, or it may be set based on a length that is effective for sleep onset, but it is not limited to this.
(d) The condition is when it is confirmed that the difference between the patient's first and second activity periods is below a threshold difference. For example, the difference could be a condition where the difference is less than or equal to a first threshold difference, or a condition where the difference is greater than or equal to a second threshold difference.
(e) The condition is when it is confirmed that at least one of the patient's measured biological sensing data satisfies a specified rule, and this can be set based on at least one biological sensing data.
(f) The condition is when a specified period has elapsed since the initiation of step (i). This period may be set as a length effective for sleep onset or based on a length that is effective for sleep onset, but it is not limited to this.

During the execution of steps (i) and (ii), at least one property of the first and second sounds in steps (iv) and (v) can be determined.

The properties of the sound to be determined can be based on the biological sensing data acquired during the previous cycle, and the determination of sound properties based on the biological sensing data can be performed using at least one rule-based system or based on the inference results of at least one artificial intelligence model.

The biological sensing data may include, but is not limited to, RR (respiratory rate), HR (heart rate), HRV, brainwaves, stress level, sounds corresponding to breathing sounds, and/or muscle tension.

The attributes may include, but are not limited to, volume, waveform, frequency, and timbre.

Steps (iv) and (v) provide the first or second sound independently of the patient's activity detection. In other words, since performing an activity is not required to provide the sound, this can be referred to as an automatic mode.

As mentioned above, after the extension of the breathing length and/or synchronization effect is implemented in steps (i) and (ii), sounds can be provided in steps (iv) and (v) regardless of activity detection. Therefore, even after the patient falls asleep while performing steps (i) to (iii), sounds for breathing guidance can continue to be provided.

If sound is provided based on the patient's activity and the sound is interrupted when the patient has not performed the activities just before sleep onset, the patient may perceive the sudden interruption of the sound, which could increase alertness. By confirming the interruption of the patient's alternating performance of activities as a condition for entering the automatic mode, sounds can be continuously provided. As a result, the increase in the patient's alertness caused by the interruption of sound can be prevented.

During the execution of steps (i) and (ii), the duration of the performance of steps (iv) and (v) can be determined. Preferably, based on the inference results of an artificial intelligence model for at least one biological sensing data, the duration of each sound's provision can be adjusted.

Steps (iv) and (v) can be provided with the same or a different cycle than the inhalation and exhalation cycle detected in steps (i) and (ii), and preferably, they can be provided with the same cycle.

The first sound and the second sound can be provided in such a way that their volume gradually increases from the start time to the maximum volume point and gradually decreases from the maximum volume point to the end time of the provision.

The time from the start time of the first and second sounds to the maximum volume point can be shorter than the time from the maximum volume point to the end time of the provision for both the first and second sounds. Preferably, the time from the start time to the maximum volume point for each of the first and second sounds can be 75% or less of the time from the maximum volume point to the end time of the provision. More preferably, the time from the start time to the maximum volume point for each of the first and second sounds can be 50% or less of the time from the maximum volume point to the end time of the provision.

The first sound and the second sound can be faded out at the end of their provision. Fade-out is a technique in music where the sound gradually decreases and ends, with the volume slowly reducing until it completely disappears.

The fade-out of the first sound can be performed so that the provision of the first sound ends after the start of the second sound, which is provided immediately after the first sound. The fade-out of the second sound can be performed so that the provision of the second sound ends after the start of the first sound, which is provided immediately after the second sound.

In step (vi), during the repetition of steps (iv) and (v), at least one property of the first sound and the second sound may change or remain the same. That is, the first sound may be the same as or at least partially different from the first sound of the previous cycle, and the second sound may be the same as or at least partially different from the second sound of the previous cycle.

The properties of the sound to be changed can be determined based on the biological sensing data acquired during the previous cycle, and the determination (or modification) of sound properties based on biological sensing data can be performed using at least one rule-based system or based on the inference results of at least one artificial intelligence model. The properties may include, but are not limited to, volume, waveform, frequency, and timbre.

The method for treatment according to the present invention aims to treat insomnia by inducing a reduction in sleep onset time and an increase in sleep duration.

The insomnia may preferably be one or more selected from the group consisting of sleep onset disorder, sleep maintenance disorder, early awakening disorder, deep sleep disorder, and circadian rhythm sleep disorder. More preferably, it may be sleep onset disorder or sleep maintenance disorder.

Sleep onset disorder refers to a condition where a person cannot fall asleep when trying to sleep, meaning that sleep onset does not occur normally. This typically means it takes a long time to fall asleep after getting into bed, or in severe cases, the person may not be able to sleep at all.

Sleep maintenance disorder refers to a condition where a person frequently wakes up during sleep or has difficulty falling back asleep after waking up. This is a problem with sleep continuity. While there may be no problem falling asleep, there are difficulties in maintaining or staying asleep.

The method for treatment of the present invention may further include a step of administering to the patient at least one of the group consisting of sleeping pills, sleep-inducing agents, and sleep aids, and preferably, this step can be performed prior to step (i).

The sleeping pills, sleep-inducing agents, and sleep aids can be administered orally or transdermally. The dosage varies depending on factors such as the patient's weight, age, gender, health condition, diet, administration time, administration method, excretion rate, and the severity of the disease. The daily dosage ranges from 0.01 to 1000 mg/kg and may be adjusted based on the administration route, severity, gender, weight, age, and other factors.

Below, specific examples and experimental examples of the present invention will be discussed.

### Example 1: Sleep Induction Experiment for Insomnia Patients

### 1-1. Experimental Design

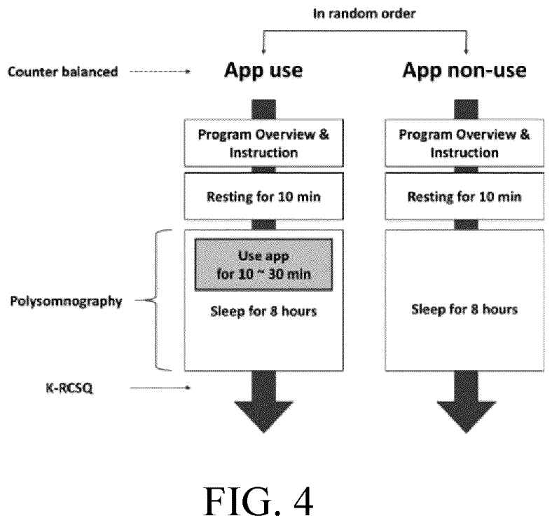

The experimental procedure is outlined schematically in FIG. 4.

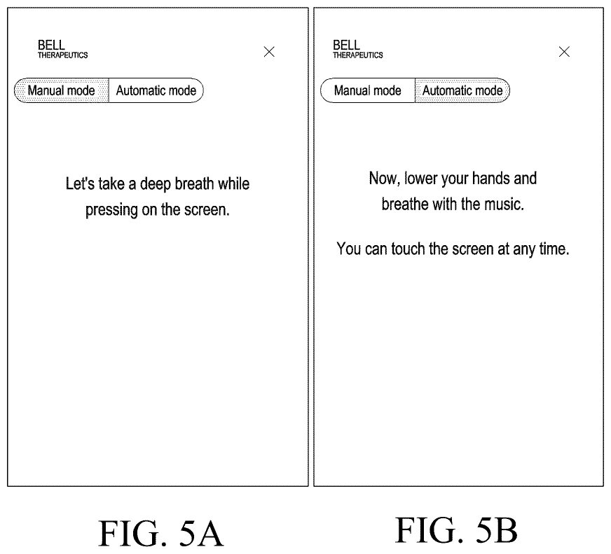

As shown in FIG. 4, the treatment method according to the present invention was provided to the subjects through a smartphone application. The application was set in two modes: manual and automatic. The subjects comfortably leaned on their beds and ran the program before falling asleep at night. The screen of the application is shown in FIGS. 5A and 5B.

As shown in FIG. 5A, the application directed the subjects to touch the screen and breathe, and the subjects naturally inhaled and exhaled in rhythm while tapping the screen according to the instructions. The application detected the screen touches and provided the first sound, and detected the release of the screen touch to provide the second sound. After two initial breaths, the application generated sounds optimized for the subject's breathing signals, guiding slower and deeper breaths.

As shown in FIG. 5B, once stable breathing was achieved, the user could tap the screen quickly to activate automatic mode, or if the subject's screen touch and release were not detected despite screen instructions, or if the icon for switching to automatic mode was tapped, or if the duration of the screen touch or release exceeded a specified threshold length, or if the difference between touch or release periods was below a threshold difference, the system would enter automatic mode and alternately provide the first sound and the second sound for a set time, independent of the subject's activities. The subjects were instructed to sleep using this treatment method. For comparison, an experiment was also conducted where the subjects were allowed to sleep without using this treatment method.

The subjects participated in the experiments under two conditions on different days, and the order of participation in the two conditions was counterbalanced to cancel out any order effects. The impact on the subjects' sleep was evaluated by comparing the results of polysomnography and the sleep quality survey (K-RCSQ).

Specifically, subjects were selected based on their experience of insomnia symptoms, referencing the DSM-V insomnia criteria. A total of 10 subjects participated. After being briefed on the procedure and details of the experiment, the subjects were given time to adapt to the sleep experiment. In this experiment, sleepwear and environmental factors (such as temperature, humidity, and lighting) were controlled to eliminate sleep-disturbing factors. The subjects visited the lab twice, each time for a one-day experiment, under the independent variable condition of ‘use/no use of the treatment method,’ and the experimental conditions were randomized to eliminate order effects. After completing the first participation, the subjects rested for at least one day before undergoing the second condition's experiment. The lab temperature was set to 20.0±2.0° C., and the humidity was 65.0±5.0%.

The subjects were 10 individuals (5 males, 5 females) in their 20s to 40s, with no other health conditions besides insomnia. All subjects self-reported requiring more than 30 minutes to fall asleep and experiencing subjective difficulty in sleep onset. The subject information is shown in Table 1 (e.g., mean and standard deviation). The subjects' usual sleep quality was assessed using the K-PSQI score, which is shown in Table 2.

TABLE 1

| Years (yrs) | Body Weight (kg) | Height (cm) | BMI(kg/m²) | K-PSQI |
|---|---|---|---|---|
| 32.2(2.9) | 75.2(11.11) | 176.20(6.77) | 24.47(3.75) | 9.5(2.15) |

The K-Pittsburgh Sleep Quality Index (K-PSQI) is a standardized self-report questionnaire used to measure an individual's sleep quality over the past month. It consists of seven components: Sleep Quality, Sleep latency, Sleep duration, Habitual sleep efficiency, Sleep Disturbances, Use of sleeping, medication, daytime dysfunction. Each component is scored on a 0 to 3 scale, with a maximum possible score of 21. A higher score indicates a higher likelihood of sleep disturbances. The interpretation of the scores is as follows:

Each component is scored on a 0 to 3 scale, with a maximum possible score of 21. A higher score indicates a higher likelihood of sleep disturbances. The interpretation of the scores is as follows:
0-4 points: Normal sleep state
5-10 points: Difficulty achieving deep sleep, with disruption in the quality and quantity of sleep
11-21 points: Sleep disturbances that interfere with daily life, requiring active treatment.

The subjects lay in bed and used the treatment method according to the present invention for 10 to 30 minutes. The sleep-inducing sound was provided through a Bluetooth speaker, and it was set to automatically stop 30 minutes after the start, even if the subject fell asleep during use. To minimize placebo effects, the product brochure or introduction provided by the manufacturer was not used in the experiment, and only objective usage instructions were provided by a researcher from the Korea Institute of Standards and Science.

### 1-2. Statistical Analysis Method

The changes in measurements under the usage condition compared to the non-usage condition were tested for statistical significance using a paired t-test. If the assumption of normality was not met, the Wilcoxon signed-rank test, a non-parametric alternative, was used. The significance level was set at 0.05. Sleep stages were analyzed through polysomnography, and the four derived metrics were compared based on the usage condition of the sleep-inducing content to evaluate its objective impact on sleep. Additionally, the overall results of the K-RCSQ survey and its five specific items were compared by usage condition to assess the subjective impact of the content on sleep quality.

### Experiment 1: Polysomnography

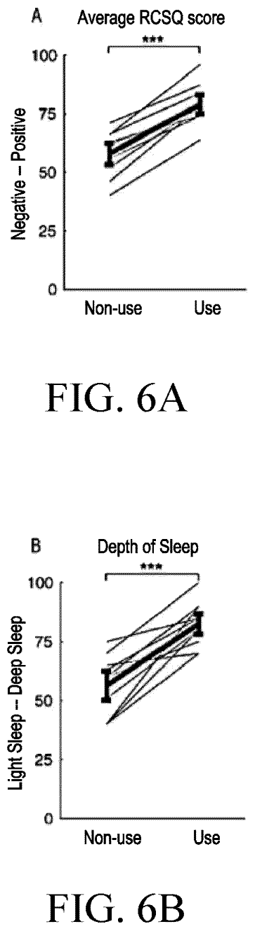

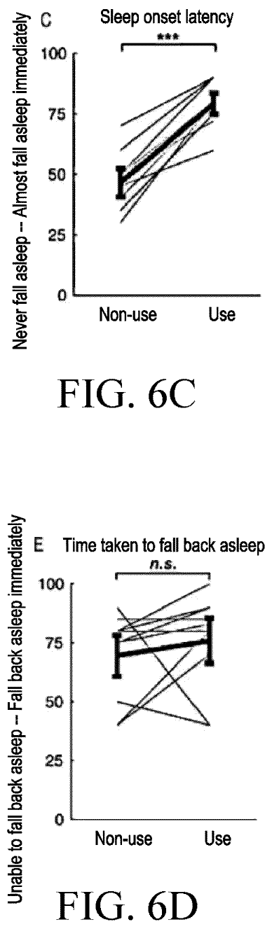

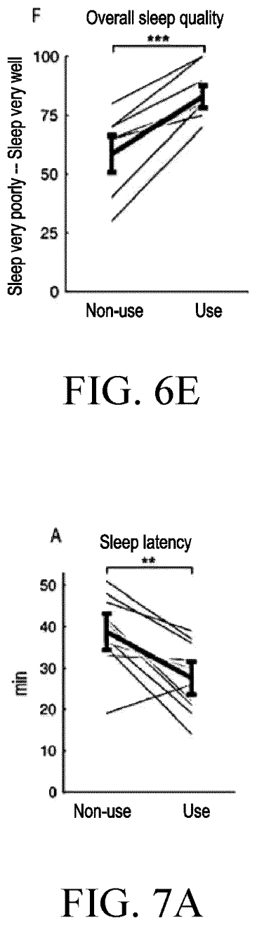

The impact of using the treatment method according to the present invention on sleep was evaluated by analyzing four key parameters through polysomnography. The results are shown in Table 2 and FIG. 6.

The four key parameters are as follows:
Sleep Latency (min): The time it takes to fall asleep. The normal range for sleep latency is between 10 to 20 minutes for healthy individuals.
Sleep Efficiency (%): The ratio of actual sleep time to the total time spent lying down and sleeping. A sleep efficiency of 80% or higher is considered normal, and healthy young adults typically have a sleep efficiency of 90% or higher.
Wake Time After Sleep Onset (WASO, min): The total amount of time spent awake during sleep.

TABLE 2

| Measurement Item | Non-user | Use | Statistic | p-value |
|---|---|---|---|---|
| sleep latency | 38.8(8.67) | 27.6(8.06) | t(9) = 3.79 | .004** |
| (min) | | | | |
| sleep | 86.7(8.96) | 95.2(2.76) | t(9) = −3.27 | .009** |
| efficiency | | | | |
| (%) | | | | |
| wake time after | 43.8(38.47) | 15.8(11.97) | t(9) = 2.89 | .18* |
| sleep onset | | | | |
| (min) | | | | |
| ratio of | 5.1(3.1) | 4.7(8.4) | Z(9) = −1.62 | .106 |
| sleep stage | | | | |

As shown in Table 2 and FIG. 6A, the treatment method significantly reduced the time taken to fall asleep (t(9)=3.79, p=0.004**). The sleep onset delay time, which was an average of 38.8 minutes under the non-use condition, decreased to an average of 27.6 minutes under the use condition, showing a reduction of 11.2 minutes. This resulted in a 28.8% reduction in sleep onset delay time compared to the non-use condition.

As shown in Table 2 and FIG. 6B, the treatment method significantly improved sleep efficiency (t(9)=−3.27, p=0.009**). Sleep efficiency is an indicator representing the ratio of actual sleep time to the total time spent lying in bed for sleep. A sleep efficiency of 80% or higher is considered within the normal range, and healthy young adults typically have a sleep efficiency of 90% or higher. The sleep efficiency, which was an average of 86.7% under the non-use condition, improved to an average of 95.2% under the use condition, showing an average improvement of 8.5%.

As shown in Table 2 and FIG. 6c, the method for treatment significantly reduced the awakening time during sleep (t(9)=2.89, p=0.018*). The average awakening time during sleep, which was reported as 43.8 minutes under the non-use condition, was reduced to an average of 15.8 minutes under the use condition, resulting in an average reduction of 28 minutes in sleep awakening time. This represents a 63.9% reduction in awakening time during sleep compared to the non-use condition.

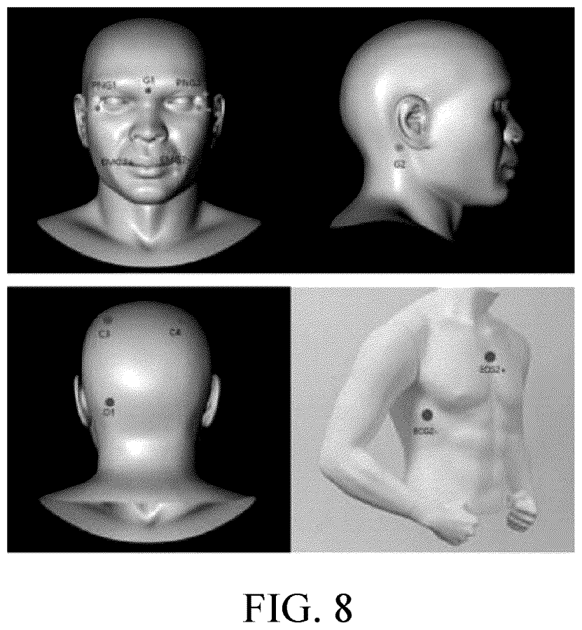

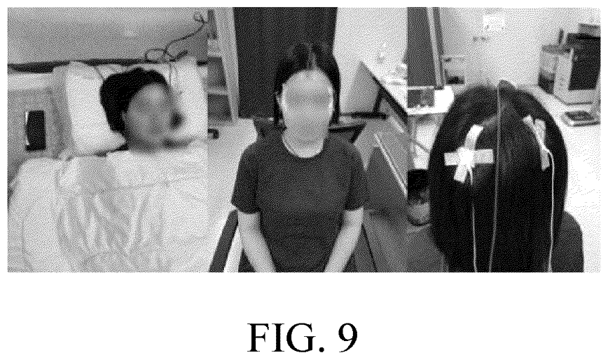

EEG sensors (C3, C4, O1 channels) were used, and the sensor locations were calculated and attached according to the 10-20 system. The sensor attachment positions and actual experimental photos are shown in FIGS. 8 and 9. EMG sensors, including PNG1+, PNG1−, PNG2+, PNG2− for eye movements and EMG2+, EMG2− for the muscles around the mouth, were used, along with ECG2+ and ECG2− channels for measuring heart potential, and G1 and G2 as the ground channels. Micromed's SystemPlus Evolution equipment was used for the measurements.

### Experiment 2: Subjective Evaluation (Sleep Questionnaire)

The sleep questionnaires used were the Korean version of the Pittsburgh Sleep Quality Index (K-PSQI) and the Korean version of the Richards-Campbell Sleep Questionnaire (K-RCSQ).

First, the K-PSQI was used to assess the participant's usual sleep quality and to screen whether they met the criteria for participation in the experiment. The results of this evaluation are shown in Table 3 below.

The K-PSQI was administered to each participant only once, while the K-RCSQ was administered twice (once for Condition 1 and once for Condition 2). Each component has a maximum score of 3 points, with a total maximum score of 21 points.

The score indicates a higher probability of experiencing sleep disturbances as it increases. (0-4 points: Normal state, 5-10 points: A state where sleep quality and quantity are disturbed, leading to difficulties in achieving restful sleep, 11-21 points: A state of sleep disturbance that interferes with daily life, requiring active treatment).

TABLE 3

| | | | | habitual | | use of | | |
|---|---|---|---|---|---|---|---|---|
| | sleep | sleep | sleep | sleep | sleep | sleeping | daytime | |
| participant | quality | latency | duration | efficiency | disturbances | medication | dysfunction | SUM |
| A | 2 | 3 | 1 | 0 | 1 | 0 | 2 | 9 |
| B | 2 | 3 | 2 | 0 | 1 | 0 | 2 | 10 |
| C | 1 | 2 | 1 | 0 | 2 | 0 | 1 | 7 |
| D | 2 | 2 | 1 | 0 | 2 | 0 | 3 | 10 |
| E | 2 | 3 | 2 | 1 | 1 | 0 | 3 | 12 |
| F | 2 | 2 | 3 | 0 | 2 | 1 | 3 | 13 |
| G | 2 | 2 | 2 | 1 | 1 | 0 | 2 | 10 |
| H | 1 | 2 | 0 | 0 | 2 | 0 | 2 | 7 |
| I | 1 | 2 | 0 | 0 | 1 | 0 | 2 | 6 |
| J | 2 | 3 | 2 | 0 | 1 | 0 | 3 | 11 |

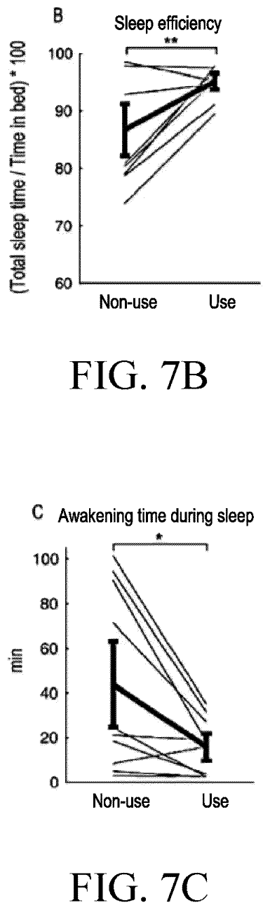

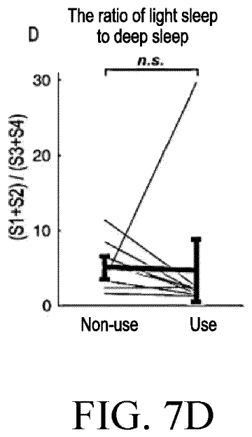

The K-RCSQ was developed to assess the quality of sleep in patients in hospitals or other medical settings, where patients self-report their sleep experiences. It evaluates five aspects of sleep: depth of sleep, time taken to fall asleep, awakenings during sleep, sleep disturbances, and overall sleep quality. The results are shown in Table 4 and FIG. 7 below. Each item is rated on a scale from 0 to 100, with higher scores indicating better sleep quality.

TABLE 4

| Measurement Item | Non-user | Use | Statistic | p-value |
|---|---|---|---|---|
| average RCSQ | 58.1(9.22) | 79.0(8.16) | t(9) = −9.76 | .000*** |
| sleep depth | 56.4(12.29) | 82.5(9.01) | t(9) = −6.07 | .000*** |
| falling asleep | 46.5(11.63) | 79.2(8.99) | t(9) = −9.29 | .000*** |
| Awakening | 59.5(8.79) | 74.7(8.99) | Z(9) = −2.82 | .005** |
| returning to sleep | 69.5(17.81) | 75.8(19.38) | t(9) = −0.81 | .441 |
| overall sleep quality | 58.5(15.34) | 79.0(8.16) | t(9) = −6.39 | .000*** |

As shown in Table 4 and FIG. 7, the treatment method significantly improved the average RCSQ score (t(9)=−9.76, p<0.000***). The average RCSQ score, which was 58.1 points under the no-treatment condition, increased to 79.0 points under the treatment condition.

When analyzing the individual components, the treatment method was effective in improving the depth of sleep as reported subjectively by the participants (t(9)=−6.07, p<0.000***). The average sleep depth score increased significantly from 56.4 points without treatment to 82.5 points with treatment. Additionally, the evaluation of sleep onset latency, i.e., the time it took to fall asleep, also showed improvement (t(9)=−9.29, p<0.000***). The average score for sleep onset latency increased from 46.5 points without the treatment to 79.2 points with the treatment.

As described above, the use of this treatment method demonstrated effectiveness in sleep induction.

### Experiment 3: Physiological Indicators (EEG) Test

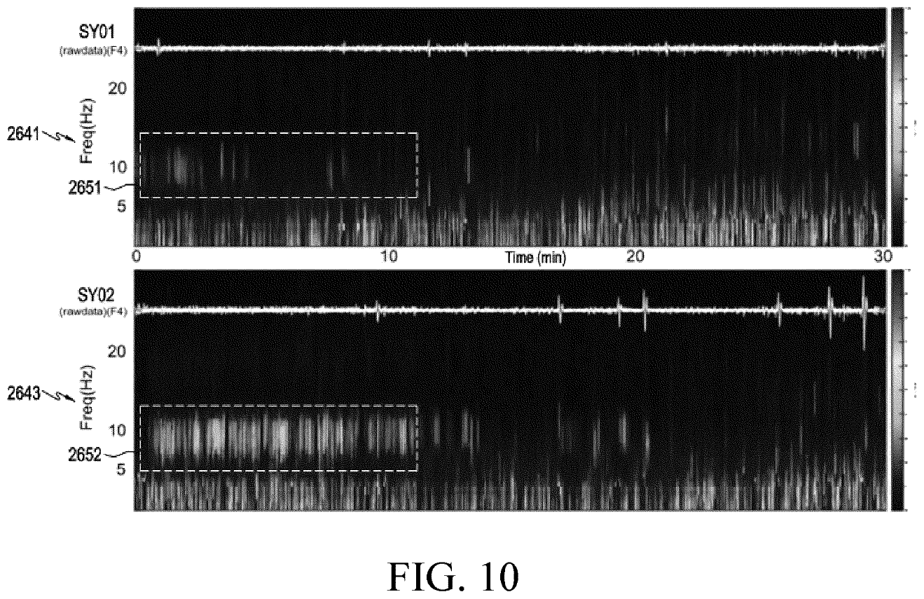

The EEG data during the intake of a sleep inducer (1st generation antihistamine, such as Doxylamine) was confirmed based on prior research, including the Phase 3 clinical trial results of Lamborexant by the FDA, and the results of this invention are shown in FIG. 10. In this figure, 2641 shows the brain waves over time during the intake of the sleep inducer (1st generation antihistamine), and 2643 shows the brain waves over time during the use of the treatment method according to this invention.

As shown in FIG. 10, the size of the alpha wave band (2651) in the 0 to 10-minute interval after administering the sleep inducer was smaller than the size of the alpha wave band (2652) in the same time interval after starting the treatment method in this invention. Alpha waves are associated with physical relaxation and sleep induction, and it was confirmed that the treatment method in this invention has a sleep-inducing effect during this period.

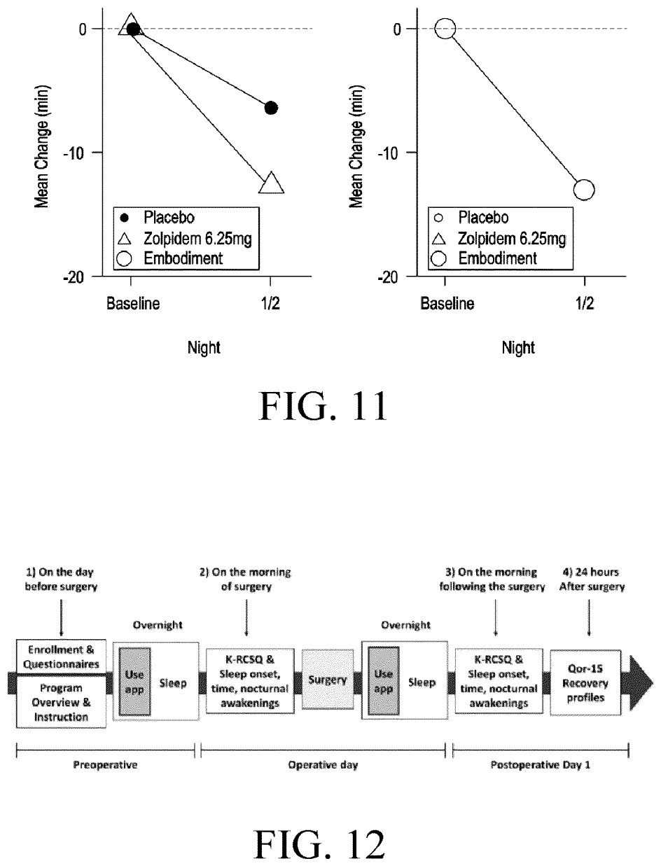

Additionally, the average change in EEG during the use of the treatment method and the intake of a sleep medication was assessed, and the results are shown in FIG. 11. In this case, the sleep medication used was zolpidem, with a dosage of 6.25 mg.

As shown in FIG. 11, compared to the average change in the latency to persistent sleep corresponding to the placebo, both the average change in the latency to persistent sleep following the intake of the sleep medication and the average change in the latency to persistent sleep during the use of the treatment method were relatively larger. Furthermore, it was confirmed that the average change during the use of the treatment method was similar to the average change observed after the intake of the sleep medication.

### Experiment 4: Induction Experiment in Breast Cancer Patients

This experiment was conducted on adult female patients under 70 years old who were scheduled for breast cancer surgery. Exclusion criteria included patients undergoing same-day or emergency surgery, patients who refused to participate, those with neurological deficits, cognitive impairments, or existing conditions related to cerebrovascular disease, as well as those with a body mass index (BMI)≥35 kg/m². Patients using sedatives or hypnotics for sleep disorders were also excluded. A total of 134 patients were screened for eligibility, and 68 patients were randomly assigned to either the experimental group or the control group after excluding 66 patients. After group assignment, 3 patients (1 from the experimental group and 2 from the control group) were excluded from the final analysis due to early discharge after surgery, which resulted in missing K-RCSQ scores. Thus, data from 65 patients were included in the final analysis, and baseline characteristics and surgical details between the two groups were similar. Both groups were provided with sleep masks and earplugs, and no additional intervention was applied to the control group.

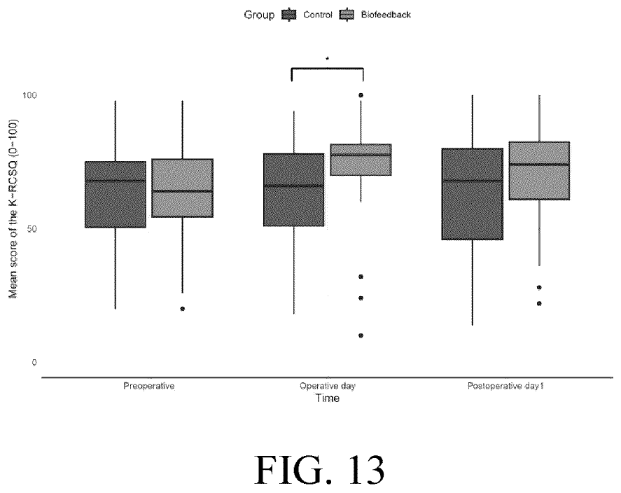

The experimental process following the treatment method of the present invention is outlined in FIG. 12.

As shown in FIG. 12, the experimental group was provided with the treatment method of the present invention via a smartphone application, similar to Example 1. The participants were instructed to use the application for at least 10 minutes before going to bed at night, and they could fall asleep while using it. The application was set to turn off automatically after a maximum of 30 minutes. Participants were instructed to use the program both the night before the surgery and the night of the surgery.

Following consent, the participants underwent four interviews at the following time points: 1) the night before surgery, 2) the morning of surgery, 3) the morning after surgery, and 4) 24 hours after surgery.

On the night before surgery, the following information was collected: patient's personal details (gender, age, medical history related to exclusion criteria), complications, medications, American Society of Anesthesiologists (ASA) physical status, Apfel score (0-4), subjective sleep disturbance, K-RCSQ, K-PSQI, QoR-15K (Korean version of the Quality of Recovery-15), and K-HADS (Hospital Anxiety and Depression Scale). Patients who scored 5 or higher on the K-PSQI were classified as having insufficient sleep.

The same information collected on the morning of the surgery was also gathered on the morning after surgery. The recovery profile during the first 24 hours post-surgery was evaluated at the 24-hour mark, which included postoperative pain assessed using an 11-point numerical rating scale (0: no pain, 10: worst imaginable pain), the use of analgesic medications, postoperative nausea and vomiting, and the quality of recovery as assessed by QoR-15K.

The average K-RCSQ scores assessed at each evaluation point are shown in FIG. 13 and Table 5.

As shown in FIG. 13, the average K-RCSQ score for sleep the night before surgery was significantly higher in the experimental group compared to the control group (experimental group: 77.5 [67.0-81.0], control group: 66.0 [54.0-78.0], P=0.049). However, no such significance was observed on the day of surgery (experimental group: 74.0 [60.0-83.0], control group: 68.0 [46.0-80.5], P=0.248).

TABLE 5

| | Biofeedback | Median | |
|---|---|---|---|
| Control group | group | difference | |
| (n = 33) | (n = 32) | (95% CI) | P value |

Evaluation of sleep on the night before surgery

| K-RCSQ mean score | 66.0 | [54.0-79.0] | 77.5 | [65.5-81.5] | 8 | (0-18) | 0.049 |
|---|---|---|---|---|---|---|---|
| (0-100) | | | | | | | |
| Sleep depth (0-100) | 60.0 | [50.0-80.0] | 80.0 | [70.0-80.0] | 10 | (0-20) | 0.017 |
| Falling asleep (0-100) | 70.0 | [55.0-85.0] | 80.0 | [60.0-90.0] | 0 | (−10-10) | 0.555 |
| Awakening (0-100) | 60.0 | [50.0-90.0] | 80.0 | [60.0; 80.0] | 10 | (0-20) | 0.161 |
| Returning to sleep (0-100) | 60.0 | [50.0-80.0] | 75.0 | [70.0-87.5] | 10 | (0-20) | 0.114 |
| Overall sleep quality | 70.0 | [50.0-80.0] | 80.0 | [70.0-90.0] | 10 | (0-20) | 0.007 |
| (0-100) | | | | | | | |
| Total sleep time, hours | 5.0 | [4.0-6.0] | 6.0 | [5.5-6.5] | 1 | (0-1) | 0.020 |
| Sleep onset latency, min # | 30.0 | [20.0-40.0] | 25.0 | [20.0-40.0] | 0 | (−10-10) | 0.787 |

Reasons for awakening during sleep

| | | | | | |
|---|---|---|---|---|---|
| Hospital environment | 26 | (86.7) | 20 | (76.9) | 0.487 |
| Anxiety | 5 | (16.7) | 4 | (15.4) | 1.000 |
| Nocturia | 6 | (20.0) | 9 | (34.6) | 0.218 |
| Others | 2 | (6.7) | 1 | (3.8) | 1.000 |

Evaluation of sleep on the day of surgery

| K-RCSQ mean score | 68.0 | [46.0-80.5] | 74.0 | [59.0-83.0] | 6 | (−4-16) | 0.248 |
|---|---|---|---|---|---|---|---|
| (0-100) | | | | | | | |
| Sleep depth (0-100) | 60.0 | [50.0-80.0] | 80.0 | [60.0-90.0] | 10 | (0-20) | 0.044 |
| Falling asleep (0-100) | 80.0 | [50.0-90.0] | 70.0 | [50.0-90.0] | 0 | (−10-10) | 0.525 |
| Awakening (0-100) | 60.0 | [50.0-85.0] | 70.0 | [50.0-80.0] | 0 | (−10-20) | 0.466 |
| Returning to sleep (0-100) | 70.0 | [45.0-90.0] | 75.0 | [52.5-80.0] | 0 | (−10-10) | 0.750 |
| Overall sleep quality | 50.0 | [35.0-80.0] | 70.0 | [60.0-90.0] | 10 | (0-30) | 0.018 |
| (0-100) | | | | | | | |
| Total sleep time, hours | 5.0 | [4.0-6.0] | 6.0 | [5.0-6.8] | 1 | (0-1) | 0.068 |
| Sleep onset latency, min | 30.0 | [20.0-30.0] | 20.0 | [20.0-37.5] | 0 | (−10-10) | 0.594 |

Reasons for awakening during sleep

| | | | | | |
|---|---|---|---|---|---|
| Hospital environment | 23 | (74.2) | 17 | (65.4) | 0.469 |
| Pain | 9 | (29.0) | 12 | (46.2) | 0.182 |
| Nocturia | 3 | (9.7) | 4 | (15.4) | 0.691 |
| Others | 2 | (6.5) | 0 | (0.0) | 0.495 |

24 hours after surgery

| | | | | | | | |
|---|---|---|---|---|---|---|---|
| QoR-15 (0-150) | 124.0 | [109.0-132.0 | 126.0 | [108-138.5] | 3 | (−7-13) | 0.545 |
| Pain intensity assessed by | 3.0 | [2.0-4.0] | 3.0 | [2.0-4.0] | 0 | (−1-1) | 0.984 |

the NRS

As shown in Table 5, among the subcomponents of the K-RCSQ, the experimental group received significantly higher scores for sleep depth and overall sleep quality in the sleep assessments conducted the night before surgery and the night of the surgery. Additionally, the experimental group reported a significantly longer subjective sleep duration on the night before surgery. However, no significant differences were observed between the two groups in the recovery profile assessed at the 24-hour mark after surgery.

Although the embodiments of the present invention have been described with reference to the accompanying drawings, it will be understood by those skilled in the art to which the present invention pertains that the invention may be carried out in other specific forms without changing the technical spirit or essential features thereof. Therefore, the embodiments described above should be understood as illustrative in all respects and not as limiting.

What is claimed is:

1. A method for treatment insomnia, the method comprising:
(i) instructing the patient to perform a first activity and inhale, detecting the patient's first activity, and providing a first sound;
(ii) instructing the patient to perform a second activity and exhale, detecting the patient's second activity, and providing a second sound;
(iii) alternately repeating steps (i) and (ii) until at least one condition is satisfied;
(iv) providing the first sound independently of detecting the patient's activity;
(v) providing the second sound independently of detecting the patient's activity; and
(vi) alternately repeating steps (iv) and (v).

2. The method of claim 1, wherein the at least one condition is satisfied when at least one of the following conditions (a) to (f) is met:
(a) failing to detect at least one of the patient's first or second activities;
(b) detecting a third activity designated for entry into step (iv);
(c) detecting that the duration of at least a portion of the patient's first or second activity exceeds a specified threshold length;
(d) confirming that the difference between the patient's first and second activity intervals is less than a critical difference;
(e) confirming that at least one measured biological sensing data from the patient satisfies a specified rule; and
(f) elapsed time exceeding a specified period since the initiation of step (i).

3. The method of claim 1, wherein the first activity causes tension in at least a portion of the patient's muscles, and/or the second activity causes relaxation of tension in at least a portion of the patient's muscles.

4. The method of claim 1,
wherein the endpoint of providing the first sound is substantially synchronized with the endpoint of detecting the first activity, and
wherein the endpoint of providing the second sound is substantially synchronized with the endpoint of detecting the second activity.

5. The method of claim 1,
wherein the endpoint of providing the first sound is after the endpoint of detecting the first activity, and
wherein the endpoint of providing the second sound is after the endpoint of detecting the second activity.

6. The method of claim 1, wherein the first sound and the second sound have a difference of at least a semitone.

7. The method of claim 1, wherein the first sound and the second sound have a difference of at least one pitch.

8. The method of claim 1,
wherein the frequency (f1) of the first sound and the frequency (f2) of the second sound satisfy the following Equation 1:
$$f 1 : f 2 = m 2 a : n 2 b , \qquad Equation 1$$
wherein, m and n are each independently 1, 3, or 5, and a and b are each independently integers from 0 to 8, with the condition that when m=n, a≠b.

9. The method of claim 1, wherein the first sound and the second sound are in a relationship of a perfect octave, perfect fifth, perfect fourth, major third, major sixth, minor third, or minor sixth.

10. The method of claim 1, wherein the first sound and the second sound each gradually increase in volume from the start of their respective provision until reaching the maximum volume, and gradually decrease in volume from the maximum volume until the endpoint of their provision.

11. The method of claim 1, wherein the first sound includes a plurality of first sub-sounds, and the second sound includes a plurality of second sub-sounds.

12. The method of claim 1, wherein steps (i) and (ii) include providing content related to bio-information along with the first sound or the second sound.

13. The method of claim 1, wherein in step (iii), at least one attribute of the first sound and the second sound is changed during the repetition of steps (i) and (ii).

14. The method of claim 1, wherein in step (iii), at least one attribute of the first sound and the second sound is maintained during the repetition of steps (i) and (ii).

15. The method of claim 1, wherein during the performance of steps (i) and (ii), at least one attribute of the first sound and the second sound in steps (iv) and (v) is determined.

16. The method of claim 1, wherein during the performance of steps (i) and (ii), the duration of the performance of steps (iv) and (v) is determined.

17. The method of claim 1, wherein steps (iv) and (v) are provided with a cycle identical to the cycle of inhalation and exhalation detected in steps (i) and (ii).

18. The method of claim 1, wherein in step (vi), at least one attribute of the first sound and the second sound is changed during the repetition of steps (iv) and (v).

19. The method of claim 1, wherein in step (vi), at least one attribute of the first sound and the second sound is maintained during the repetition of steps (iv) and (v).

20. The method of claim 1, wherein the method is intended to reduce sleep onset time and increase sleep duration.

21. The method of claim 1, wherein the insomnia is one or more selected from the group consisting of sleep onset disorder, sleep maintenance disorder, early awakening disorder, deep sleep disorder, and circadian rhythm sleep disorder.

22. The method of claim 1, further comprising:
the step of administering to the patient at least one selected from the group consisting of sleeping pills, sleep inducer, and sleep aids.

23. The method of claim 1, wherein the sleeping pills, sleep inducer, and sleep aids are administered orally or transdermally.

24. The method of claim 1, further comprising:
the step of administering an insomnia digital therapy based on CBT (Cognitive Behavioral Therapy) to the patient.

25. The method of claim 1,
wherein the first activity is an activity intentionally performed by the patient independently of the biological signs caused by the inhalation, or
wherein the second activity is an activity intentionally performed by the patient independently of the biological signs caused by the exhalation.
